(19) United States
(12) Patent Application Publication
(10) Pub. No.: US 2006/0121108 A1
(43) Pub. Date: Jun. 8, 2006

(54) System and method for producing a directly compressible, high-potency formulation of metformin hydrochloride

(76) Inventor: C. Krishna Prasad, Hyderabad (IN)

Correspondence Address: LAMORTE & ASSOCIATES P.C., P.O. BOX 434, YARDLEY, PA, 19067, US

(21) Appl. No.: 11/000,878

(22) Filed: Dec. 2, 2004

(51) Int. Cl. A61K 9/20; A61K 31/155

(52) U.S. Cl. 424/464; 514/635

(57) ABSTRACT

The formulation and method of producing a directly compressible pharmaceutical composition that has metformin as an active ingredient. To produce the pharmaceutical composition, a binding solution of povidone, starch and water is created. The binding solution is then mixed with a high concentration of metformin HCl to form a first mix composition. The first mix composition is dried. The dried first mix composition is then milled into granules. Various dry lubricants can be blended with the granules if desired. The result is a bulk pharmaceutical composition in which metformin HCl is the active ingredient and is present in a concentration of over 90% by weight. Yet, the bulk pharmaceutical composition exists as a free flowing powder that can be directly compressed into tablets and is chemically stable during periods of long storage.

### BACKGROUND OF THE INVENTION

1. Field of the Invention

The present invention relates to the methodologies and formulations used in the creation of metformin hydrochloride tablets.

2. Background Art

Metformin hydrochloride, known as metformin HCL, is a pharmaceutical compound that is widely used in the treatment of diabetes. Metformin HCl is typically administered orally, wherein it is taken by a patient in the form of a tablet.

A problem associated with metformin HCL is that metformin HCL, itself, cannot be readily compressed into a tablet. The particles of powderized metformin HCL tend not to bond together under compression forces. Consequently, in order to form the metformin HCl into a tablet, the metformin HCL must be mixed with some other inert excipient that will bind the metformin HCl when in a tablet press.

There are certain disadvantages that occur when metformin HCl is mixed with traditional excipients. One of the disadvantages is that of pill size. Many physicians recommend dosages of Metformin HCl to their patients of between 500 mg to 1000 mg. Many patients prefer to take a single pill in a dosage. As a consequence, pharmaceutical companies may attempt to place up to 1000 mgs of metformin HCl into a single tablet. Since metformin HCL does not compress well, it does not reduce much in volume when compressed. A large tablet size is therefore required to hold 1000 mgs of metformin HCL. The addition of excipients requires even larger tablets be used. The end result is often an overly large “horse pill” that is too large for many patients to swallow. Thus, many pharmaceutical companies only form smaller, lower dosage tablets.

In the prior art, there are many binding compounds that have been mixed with metformin HCl that enable the metformin HCl to be formed into a tablet. Such prior art compositions are exemplified by U.S. Pat. No. 6,117,451 to Kumar, entitled Direct Compression Metformin Hydrochloride Tablets and U.S. Pat. No. 6,524,618 to Kumar, entitled Directly Compressible Extended-Release Matrix Formulation For Metformin Hydrochloride. In both these patents, the metformin HCl is mixed with up to 20% methylcellulose, and up to 10% other excipients. Thus, the final composition contains only 70% of the metformin HCl. As a result, up to 30% of the tablet is occupied by an inert excipient. The resulting tablet is therefore 30% larger than it needs to be.

Another disadvantage of making tablets from metformin HCl is the fact than many of the excipients that are traditionally added to the metformin HCl must be added to the metformin HCl just prior to the formation of tablets. For instance, many traditional excipients have high moisture levels. This helps the excipients mix with the metformin. The excess moisture is expelled shortly after mixing during the tablet formation process. However, if the excipients were added to the metformin HCl long before the tableting process, the moisture could react with the metformin HCl and could have an adverse effect on the quality of the metformin HCl. The combination could also adversely effect the powder flow characteristics of the metformin HCl, thereby making the metformin hard to store and handle in bulk.

In the pharmaceutical industry, more and more active pharmacological materials are being prepared off-site in bulk. These bulk materials may be created several weeks to several months before they are actually used in the formation of a pharmaceutical tablet. The bulk material is shipped to factories where the bulk material is compressed into tablets and packaged. It is preferred that the bulk material be delivered to the tablet factory in a condition that is directly ready for tabletization without the need for premixing or other processing.

A need therefore exists for a formulation of metformin HCl that is directly compressible, yet can be created many months prior to the formation of tablets without adverse effects on the quality of the metformin HCl or the ability of the metformin HCl to be shipped and stored as a powdered bulk material. This need is met by the present invention as described and claimed below.

### SUMMARY OF THE INVENTION

The present invention is a method of producing a directly compressible pharmaceutical composition that has metformin as an active ingredient, and the pharmaceutical composition itself. To produce the pharmaceutical composition, a binding solution of povidone, starch and water is created. At least two different molecular weights of the povidone are used in the binding solution. The binding solution is then mixed with a high concentration of metformin HCl to form a first mix composition.

The first mix composition is dried to a moisture content of less than 2% by weight. Once dried, the first mix composition contains over 90% metformin HCl by weight. The dried first mix composition is then milled into granules. Various dry lubricants can be blended with the granules if desired. The result is a bulk pharmaceutical composition in which metformin HCl is the active ingredient and is present in a concentration of over 90% by weight. Yet, the bulk pharmaceutical composition exists as a free flowing powder that can be directly compressed into tablets and is chemically stable during periods of long storage.

### BRIEF DESCRIPTION OF THE DRAWINGS

For a better understanding of the present invention, reference is made to the following description of an exemplary embodiment thereof, considered in conjunction with the accompanying drawing, in which:

FIG. 1 is a schematic that illustrates the method of manufacture of the present invention pharmaceutical composition.

### DETAILED DESCRIPTION OF THE DRAWINGS

The present invention is a formulation and method for making a bulk pharmaceutical composition in which metformin HCl is the active ingredient and is present in a concentration of over 90% by weight. Yet, the bulk pharmaceutical composition exists as a free flowing powder that can be directly compressed into tablets and is chemically stable during long storage.

The present invention pharmaceutical composition uses a small amount of starch to bind the metformin HCl. To help the starch bind the metformin HCl, different molecular weights of povidone are added as binding catalysts.

An exemplary composition of pharmaceutical composition is shown by the following table.

TABLE 1

| Percent Weight | Ingredient |
|---|---|
| 90%-97% | Metformin HCl |
| 0.5%-2.5% | Povidone (K-30) |
| 0.5%-3.0% | Povidone (K-90) |
| 0.5%-2.0% | Pregelatinized Starch |
| 0.1%-0.5% | Crospovidone |
| 0.1%-1.0% | Magnesium Stearate |

The methodology of how to mix and process these beginning ingredients to produce the present invention is best explained by reference to the drawings.

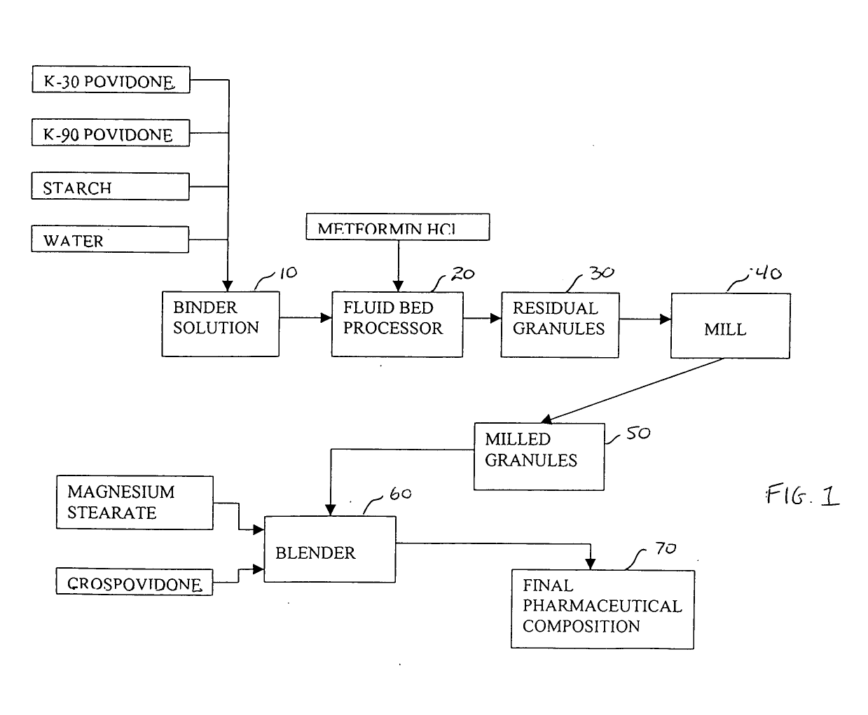

Referring to FIG. 1, it can be seen that a binder solution 10 is created by stirring together the starter amounts of K-30 povidone, K-90 povidone and the pregelatinized starch in a stainless steel vessel. The K-30 povidone and K-90 povidone are variants of povidone that have different molecular weights. Povidone is 1-ethenyl-2-pyrrolidinone homopolymer and is sold commercially under the tradenames Plasdone, Periston and Protangent.

The pregelantized starch is a carbohydrate polymer containing amylose and amylopectin in varying amounts depending upon the base plant source of the starch. It will be understood that any such starch, such as those derived from corn, wheat, rice and potatoes can be used.

A volume of purified water is added to the binder solution 10 in an amount sufficient to fully dissolve the K-30 povidone and the K-90 povidone. Although the amounts of K-30 povidone and K-90 povidone can be varied within the ranges set forth in Table 1, it is preferred that close to equal quantities of both molecular weights of the povidone be used. The use of different molecular weights enables the povidone to better bind to the metformin HCl and to the pregelentinized starch. The povidone binds to the metformin HCl and acts as a binding catalyst for the pregelentinized starch. As a result, although only a small volume of pregelentinized starch is used, it is capable of binding all of the metformin HCl that eventually will be mixed with the binder solution.

The binder solution 10 is spray mixed with the metformin HCl within a commercial fluid bed processor 20. The temperature within the fluid bed processor 20 is maintained between 30 degrees Celsius and 45 degrees Celsius. The atomizing air pressure within the fluid bed processor 20 is preferably kept between three and five bar during spraying.

The fluid bed processor 20 uniformly mixes the metformin HCl and the binder solution 10. The purified water that was initially added to the binder solution 10 is evaporated out. This leaves the metformin HCl and binder materials as dry residual granules 30 with a moisture content of between one percent and one and a half percent, by weight.

After being dried to the desired moisture contents within the fluid bed processor 20, the residual granules 30 are removed and taken to a mill 40. The residual granules 30 are then sifted and milled to a desired particle size. This produces milled granules 50.

The milled granules 50 are then added to a blender 60. The crospovidone and magnesium stearate are added to the blender 60 in the amounts listed in Table 1. The blender 60 is then run to create a homogenous mixture. The crospovidone and magnesium stearate are added as dry lubricants, thereby preventing biding between granules prior to tabletization. It will be understood that the use of crospovidone and magnesium stearate are selected because of their lubrication properties and their ability to remain inert in the composition. Other lubricants however, can be used.

When the blender 60 is emptied, the final pharmaceutical composition 70 is complete. The resulting pharmaceutical composition 70 has a potency of up to 97% metformin HCl, by weight. Yet the pharmaceutical composition 70 can be directly compressed into tablets without any further processing. Using standard tablet presses and compression forces, a tablet produced from the final pharmaceutical composition 70 would have a density of between 0.7 and 0.9 grams per milliliter. The tablet would be well formed having industry acceptable degrees of hardness, chip resistance and disintegration time.

The excipients mixed with the metformin HCl within the final pharmaceutical composition 70 are inert to the metformin HCl. Thus, the quality and potency of the metformin HCl remains unaffected by the excipients. The final pharmaceutical composition 70 can therefore be stored in bulk for long periods of time prior to use. Once the final pharmaceutical composition 70 is to be used, the pharmaceutical composition 70 can be directly loaded into a tabletization press without the need of any mixing or any other preparatory processing.

It will be understood that a person skilled in the art can make variations to the described embodiment of the pharmaceutical compound by varying ingredient proportions and substituting chemically equivalent compounds. For example, in the pharmaceutical industry, there are many types of binding starches. A person skilled in the art can substitute some other binding starch for the pregelatinized starch stated. All such variations, modifications and alternate embodiments are intended to be included within the scope of the present invention as set forth by the claims.

What is claimed is:

1. A pharmaceutical composition that is directly compressible into tablet form, comprising:
90% to 97% by weight of metformin HCl;
1% to 5.5% by weight povidone; and
0.5% to 2% by weight starch.

2. The composition according to claim 1, wherein said povidone includes two separate molecular weights of povidone.

3. The composition according to claim 2, wherein said povidone includes K-30 povidone and K-90 povidone.

4. The composition according to claim 3, wherein said K-30 povidone is present between 0.5% and 2.5% by weight.

5. The composition according to claim 4, wherein said K-90 povidone is present between 0.5% and 3.0% by weight.

6. The composition according to claim 1, wherein said starch is a pregelentinized starch.

7. The composition according to claim 1, further including between 0.1% and 1.5% of at least one dry lubricant.

8. The composition according to claim 1, further including between 0.1% and 1.0% magnesium stearate.

9. The composition according to claim 1, further including between 0.1% and 0.5% crospovidone.

10. A method of creating a directly compressible pharmaceutical composition that has metformin HCl as an active ingredient, comprising the steps of:
creating a binding solution of povidone, starch and water;
mixing said binding solution with metformin HCl to form a first composition;
drying said first composition to a moisture content of less than 2% by weight, wherein said first composition contains over 90% metformin HCl by weight; and
milling said first composition into granules.

11. The method according to claim 10, wherein said step of creating a binding solution includes combining two separate molecular weights of povidone with the starch and enough water to dissolve both the povidone and the starch.

12. The method according to claim 11, wherein said separate molecular weights of povidone include K-30 povidone and K-90 povidone.

13. The method according to claim 10, wherein said step of mixing said binding solution with said metformin HCl includes mixing said binding solution with said metformin HCl in a fluid bed processor.

14. The method according to claim 10, further including the step of blending said granules with a dry lubricant.

15. The method according to claim 10 further including the step of blending said granules with magnesium stearate.

16. The method according to claim 15, further including the step of blending said granules with crospovidone.
